(19) United States
(12) Patent Application Publication
(10) Pub. No.: US 2024/0315939 A1
(43) Pub. Date: Sep. 26, 2024

(54) METHOD TO PREVENT OXIDATIVE STRESS IN THE SKIN OF THE SCALP THROUGH THE COMBINATION OF L-ASCORBIC ACID WITH ANTI-FUNGAL AGENTS

(71) Applicant: KilgourMD, Inc., Wilmington, DE (US)

(72) Inventor: James M. Kilgour, Menlo Park, CA (US)

(21) Appl. No.: 18/535,563

(22) Filed: Dec. 11, 2023

Related U.S. Application Data
(60) Provisional application No. 63/454,222, filed on Mar. 23, 2023.

(51) Int. Cl. A61K 8/368; A61K 8/14; A61K 8/49; A61K 8/67; A61Q 19/00

(52) U.S. Cl. CPC A61K 8/368; A61K 8/14; A61K 8/4926; A61K 8/676; A61Q 19/00

(57) ABSTRACT

A method to prevent oxidative stress in the skin of the scalp through the combination of L-Ascorbic acid with anti-fungal agents in a cosmetic preparation for the scalp is disclosed. The addition of anti-fungal agents (such as zinc pyrithione or piroctone olamine) to L-Ascorbic acid allows the cosmetic preparation to specifically treat the unique drivers of oxidative stress (and therefore extrinsic skin ageing) within the scalp.

### CROSS REFERENCE TO RELATED APPLICATIONS

The present application claims priority to U.S. Provisional Patent Application Ser. No. 63/454,222, filed on Mar. 23, 2023, the contents of which are incorporated herein by reference.

### FIELD OF THE INVENTION

The present invention relates to a cosmetic preparation method for preventing oxidative stress in the skin of a scalp (human). Further, the present invention relates to a topical cosmetic composition comprising L-ascorbic acid and anti-fungal agents.

### BACKGROUND OF THE INVENTION

Oxidative stress for people is caused within the skin by exposure to harmful agents in the environment such as ultraviolet (UV) radiation and pollution. This environmental stress is known to be the primary driver of extrinsic skin ageing, which in itself accounts for 80% of skin ageing. The scalp is a special site on the skin surface, made up of terminal hairs produced by hair follicle stem cells. Over time, oxidative stress can lead to the ageing of the scalp and contribute to hair loss. The scalp in most people is colonized by a commensal fungus called Malassezia, which in some individuals can produce a disease known as seborrheic dermatitis (colloquially known as dandruff). Even in individuals without clinically evident seborrheic dermatitis, Malassezia is known to still contribute to oxidative stress within the special site of the scalp.

No other cosmetic preparation includes a high potency topical antioxidant, specifically L-Ascorbic acid, combined with anti-fungal agents in a cosmetic preparation specifically designed for application to the scalp. This results in other cosmetic preparations inadequately treating the specific drivers of oxidative stress and extrinsic skin ageing within the special site of the scalp.

The addition of anti-fungal agents (such as zinc pyrithione and piroctone olamine) to L-Ascorbic acid allows the cosmetic preparation to specifically treat the unique drivers of oxidative stress (and therefore extrinsic skin ageing) within the scalp.

### SUMMARY OF THE INVENTION

One aspect of the present invention relates to a cosmetic preparation method for a topical cosmetic composition to prevent oxidative stress in skin of a scalp. The method comprises a step of providing a L-ascorbic acid and an anti-fungal agent. The anti-fungal agent includes a first anti-fungal agent selected from a group consisting of zinc pyrithione, piroctone olamine, and a combination thereof. The method further comprises a step of mixing the L-ascorbic acid and the anti-fungal agent in a vehicle.

Another aspect of the present invention relates to a topical cosmetic composition comprising L-ascorbic acid and an anti-fungal agent. The anti-fungal agent includes a first anti-fungal agent selected from a group consisting of zinc pyrithione, piroctone olamine, and a combination thereof. The cosmetic composition is suitable for cosmetic application to prevent oxidative stress in skin of a scalp (human).

### BRIEF DESCRIPTION OF THE DRAWINGS

FIG. 1 demonstrates the molecular structure of L-Ascorbic acid;

FIG. 2 demonstrates the molecular structure of zinc pyrithione; and

FIG. 3 demonstrates the molecular structure of piroctone olamine.

These three figures demonstrate the three molecular structures which may be either combined together or used individually to form the cosmetic preparation for the scalp.

### DETAILED DESCRIPTION OF THE INVENTION

As stated above, oxidative stress on the skin is caused by exposure to harmful agents in the environment such as ultraviolet (UV) radiation and pollution. This environmental stress is known to be the primary driver of extrinsic skin ageing, which in itself accounts for 80% of skin ageing. The scalp is a special site on the skin surface, made up of terminal hairs produced by hair follicle stem cells. Over time, oxidative stress can lead to ageing of the scalp and contribute to hair loss. The scalp in most people is colonized by a commensal fungus called Malassezia, which in some individuals can produce a disease known as seborrheic dermatitis (colloquially known as dandruff). Even in individuals without clinically evident seborrheic dermatitis, Malassezia is known to still contribute to oxidative stress within the special site of the scalp. The present invention claimed here solves this problem.

According to one embodiment of the present invention, a cosmetic preparation method comprises a process of combining the high potency topical antioxidant L-Ascorbic acid (the most biologically active form of Vitamin C) with known anti-fungal agents, including zinc pyrithione and piroctone olamine. This combined composition functions to neutralize oxidative stress within the special skin site of the scalp. The L-Ascorbic acid functions to neutralize oxygen free radicals while the anti-fungal agents reduce the oxidative stress burden contributed to by the Malassezia.

The process of present invention differs from what currently exists. While cosmetic preparations of L-Ascorbic acid exist for application to the skin, no current formulation combines the L-Ascorbic acid with the anti-fungal agents (such as zinc pyrithione and piroctone olamine) to specifically treat the unique drivers of oxidative stress (and therefore extrinsic ageing) of the scalp.

Thus, the present invention is an improvement on what currently exists. While cosmetic preparations of L-Ascorbic acid exist for application to the skin, no current formulation combines the L-Ascorbic acid with anti-fungal agents (such as zinc pyrithione and piroctone olamine) to specifically treat the unique drivers of oxidative stress (and therefore extrinsic ageing) of the scalp.

The lack of combining anti-fungal agents with a potent topical antioxidant (specifically L-Ascorbic acid) results in an inferior treatment of oxidative stress, and therefore extrinsic skin ageing, within the special skin site of the scalp.

The addition of anti-fungal agents (such as zinc pyrithione and piroctone olamine) to L-Ascorbic acid allows the cosmetic preparation to specifically treat the unique drivers of oxidative stress, and therefore extrinsic skin ageing, within the scalp.

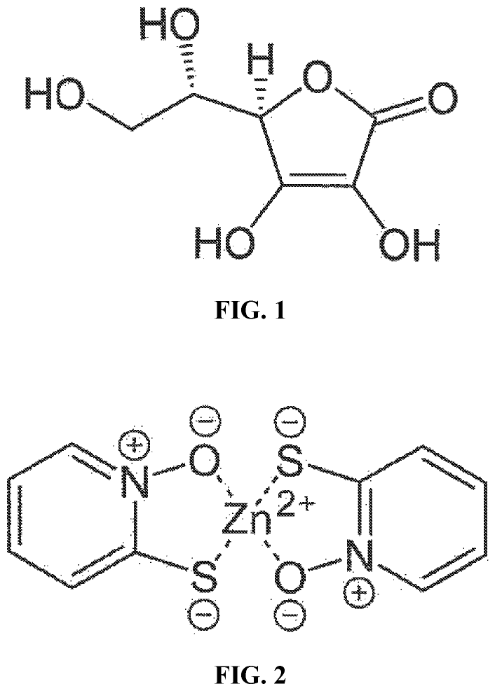

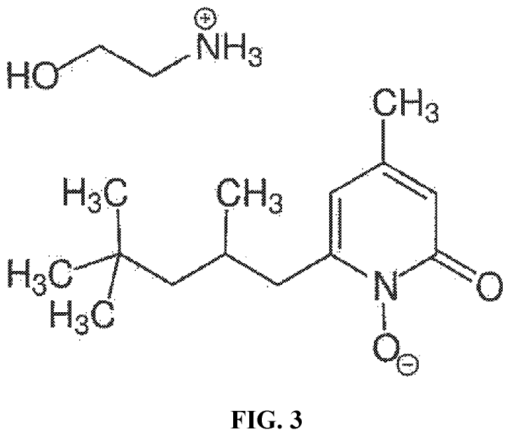

Referring to FIGS. 1-3, FIG. 1 demonstrates the molecular structure of L-Ascorbic acid; FIG. 2 demonstrates the molecular structure of Zinc pyrithione; and FIG. 3 demonstrates the molecular structure of piroctone olamine.

According to another embodiment, all of the three molecules demonstrated in FIGS. 1-3 are combined together to formulate a topical cosmetic composition for the scalp. The cosmetic preparation is conducted by combining the molecules described herein and demonstrated in FIGS. 1-3 alongside a suitable vehicle. In a typical preparation procedure, zinc pyrithione and piroctone olamine are mixed initially into a vehicle at a temperature of 40 C until uniform. Then L-ascorbic acid is added in and mixed until uniform. The final state of the cosmetic composition is serum.

The process of combining the three molecules illustrated in FIGS. 1-3 results in a unique and novel cosmetic preparation designed to specially suit application to the scalp. This results in a cosmetic preparation uniquely able to target the drivers of oxidative stress, and extrinsic skin ageing, within this special site of the skin.

The necessary elements are the combination of L-Ascorbic acid with at least one known anti-fungal agent, such as zinc pyrithione (FIG. 2) or piroctone olamine (FIG. 3). Combination of the L-Ascorbic acid with either solely the zinc pyrithione (FIG. 2), or the piroctone olamine (FIG. 3), or another suitable anti-fungal agent, would lead to substantially the same efficacy of the cosmetic preparation.

Yet, according to another embodiment, a liposome is used to incorporate the L-ascorbic acid to form a liposomal encapsulated L-ascorbic acid before mixing the encapsulated L-ascorbic acid with the anti-fungal agents and other ingredients in a vehicle. In a preparation procedure, the ingredients, including a vehicle, are weighed into a container. The container is heated to 60 C while the content in the container is stirred gently using a stick blender until all the ingredients are fully dissolved. The mixture is cooled to 40 C with gentle stirring. The anti-agent(s) are added one by one to the vehicle and mixed well until uniform. Then, L-ascorbic acid is mixed with liposome in a separate beaker before being added to the above mixture. The overall mixture in the container is mixed well until uniform. The pH of the content in the container is tested using a pH meter. 50% citric acid solution or 50% sodium hydroxide solution may be added drop by drop to adjust the pH if need.

L-Ascorbic acid is used in this cosmetic preparation preferably at a concentration (per International Nomenclature of Cosmetic Ingredients (INCI) convention) between 5% to 40%. Zinc pyrithione is included at a concentration preferably between 0.1% to 0.25% (INCI convention). Piroctone olamine is included at a concentration preferably between 0.1% to 1% (INCI convention). While these concentration ranges represent the preferred ranges, concentrations outside of them would likely still have substantially the same efficacy.

In addition to the above, other anti-fungal agents could be included in this cosmetic preparation. The other anti-fungal agents include, but are not limited to, coal tar, salicylic acid (preferably at a concentration between 0.5% to 4%), selenium sulfide, sulfur, the Azole antifungals (including but not limited to ketoconazole), and terbinafine.

The topical cosmetic composition would be applied at least once daily directly to the scalp.

While the present invention is provided in detail in connection with only a limited number of anti-fungal agents in the topical cosmetic composition, it should be readily understood that the invention is not limited to such disclosure. Rather, the present invention can be modified to incorporate any number of anti-fungal agents, additives, derivatives, or to have the preparation method conducted alongside various suitable vehicles, which are not heretofore described, but are commensurate with the spirit and scope of the present invention. Additionally, while a limited number of embodiments have been described, it is to be understood that the exemplary embodiments may include only some of the described exemplary aspects. Accordingly, the present invention is not to be seen as limited by the foregoing description but is only limited by the scope of the appended claims.

What is claimed is:

1. A cosmetic preparation method for a topical cosmetic composition to prevent oxidative stress in skin of a scalp, the method comprising:
providing a L-ascorbic acid;
providing an anti-fungal agent, wherein the anti-fungal agent comprise a first anti-fungal agent selected from a group consisting of zinc pyrithione, piroctone olamine, and a combination thereof; and
mixing the L-ascorbic acid and the anti-fungal agent in a vehicle.

2. The method of claim 1, further comprising:
providing a liposome; and
incorporating the L-ascorbic acid into the liposome to form a liposomal encapsulated L-ascorbic acid before mixing the encapsulated L-ascorbic acid with the anti-fungal agent.

3. The method of claim 1, wherein the anti-fungal agent further comprises a second anti-fungal agent selected from a group including coal tar, salicylic acid, selenium sulfide, sulfur, Azole antifungals, terbinafine, and a combination thereof.

4. The method of claim 3, wherein the Azole antifungals include ketoconazole.

5. The method of claim 1, wherein the L-Ascorbic acid in the composition is at a concentration range between 5% and 40% based on per INCI convention.

6. The method of claim 1, wherein the zinc pyrithione in the composition is at a concentration range between 0.1% and 0.25%.

7. The method of claim 1, wherein the piroctone olamine in the composition is at a concentration range between 0.1% and 1%.

8. The method of claim 3, wherein the salicylic acid in the composition is at a concentration range between 0.5% to 4%.

9. A topical cosmetic composition, comprising L-ascorbic acid and an anti-fungal agent, wherein the anti-fungal agent includes a first anti-fungal agent selected from a group consisting of zinc pyrithione, piroctone olamine, and a combination thereof.

10. The composition of claim 9, wherein the anti-fungal agent further comprises a second anti-fungal agent selected from a group including coal tar, salicylic acid, selenium sulfide, sulfur, Azole antifungals, terbinafine, and a combination thereof.

11. The composition of claim 9 further comprising a liposome, wherein the liposome incorporates the L-ascorbic acid to form a liposomal encapsulated L-ascorbic acid.

12. The composition of claim 10, wherein the Azole antifungals include ketoconazole.

13. The composition of claim 9, wherein the L-Ascorbic acid in the composition is at a concentration range between 5% and 40% based on per INCI convention.

14. The composition of claim 9, wherein the zinc pyrithione in the composition is at a concentration range between 0.1% and 0.25%.

15. The composition of claim 9, wherein the piroctone olamine in the composition is at a concentration range between 0.1% and 1%.

16. The composition of claim 10, wherein the salicylic acid in the composition is at a concentration range between 0.5% and 4%.
